(19) United States
(12) Patent Application Publication
(10) Pub. No.: US 2017/0189000 A1
(43) Pub. Date: Jul. 6, 2017

(54) APPARATUS AND METHODS FOR SEALING A VASCULAR PUNCTURE

(71) Applicant: ACCESS CLOSURE, INC., SANTA CLARA, CA (US)

(72) Inventors: ALI ALMEDHYCHY, Palo Alto, CA (US); JOHN FREDERIC MAGOLSKE, Mountain View, CA (US)

(73) Assignee: ACCESS CLOSURE, INC., SANTA CLARA, CA (US)

(21) Appl. No.: 15/398,051

(22) Filed: Jan. 4, 2017

Related U.S. Application Data
(60) Provisional application No. 62/274,585, filed on Jan. 4, 2016.

(51) Int. Cl. A61B 17/00

(52) U.S. Cl. CPC A61B 17/0057; A61B 2017/00659; A61B 2017/00623; A61B 2017/00004

(57) ABSTRACT

A vascular closure apparatus is described having an elongate shaft, an atraumatic distal tip and a bioabsorbable sealant for sealing an arteriotomy, and methods of use thereof. The distal tip can comprise a plurality of deflectable panels through which the sealant can be ejected. The apparatus is positioned within the vessel puncture tract without the aid of a guidewire and advanced until the smooth applicator tip is in close proximity or in contact with the arteriotomy lips. An inner tamping member may be manipulated relative to an outer sheath member to release the sealant at the arteriotomy site. An exemplary embodiment includes releasing the sealant onto a mechanically closed arteriotomy having one or more spaces or gaps therein, such that the sealant performs as an adjunctive closure. In situ, the sealant can transform to a flowable or gel state and cover the cracks and spaces along the mechanically closed arteriotomy.

### FIELD

Medical devices, and more particularly, medical devices and methods designed for percutaneous vascular closure for sealing a vascular puncture extending through tissue to a blood vessel.

### BACKGROUND

Apparatus and methods are known for accessing a patient's vasculature percutaneously, e.g., to perform a procedure within the vasculature, and for sealing the puncture that results after completing the procedure. For example, a hollow needle may be inserted through a patient's skin and overlying tissue into a blood vessel. A guide wire may be passed through the needle lumen into the blood vessel, whereupon the needle may be removed. An introducer, procedural or femoral sheath may then be advanced over the guide wire into the vessel, e.g., in conjunction with or subsequent to one or more dilators. A catheter or other device may be advanced through the introducer sheath and over the guide wire into a position for performing a medical procedure. Thus, the introducer sheath may facilitate accessing and/or introducing various devices into the vessel, while minimizing trauma to the vessel wall and/or minimizing blood loss.

Wounds such as arteriotomies can arise in the blood vessel from these various medical procedures, especially for blood vessels acting as sites for catheter insertion during diagnostic and/or interventional catheterization. After such procedures have been completed, the arteriotomy that was created as an access point during the medical procedure needs to be closed.

One method used to stop the bleeding from a puncture or arteriotomy can be by application of pressure to the wound site, such as by applying external pressure to the overlying tissue either manually or by applying sandbags, until hemostasis occurs. This procedure, however, may be time consuming and expensive, requiring as much as an hour of a medical professional's time. It is also uncomfortable for the patient, and may require the patient to remain immobilized in the operating room, catheter lab, or holding area. In addition, a risk of hematoma exists from bleeding before hemostasis occurs.

Various devices have been suggested for sealing vascular punctures resulting from such procedures, however, these various closure devices tend to rely on either purely mechanical or purely biological means to close the wound. While effective in small bore closures, e.g., arteriotomies that are sized at less than about 8 French, the use of a single closure means may not be adequate when closing a large bore, e.g., an arteriotomy sized at greater than 8 French. As is understood by one of skill in the art, the French size is based on the size of the introducer sheath that makes the puncture or arteriotomy.

For example, US Patent Publication No. 2008/0249545 to Shikhman et al. describes a mechanical based device. The Shikhman publication describes a percutaneous surgical device, which comprises a combination wound suturing and crimping and cutting device. In one exemplary embodiment a crimping and cutting device portion nests within a suturing device portion. The combined device may locate a vessel wound and pass suture through the vessel walls surrounding the wound. Then, the crimping and cutting portion may detach, the suturing portion may be removed, and the crimping and cutting portion may be located to the wound site to apply a fastener (e.g., a ferrule).

U.S. Pat. No. 7,060,078 to Hathaway et al. describes another mechanical based device. The '078 patent describes a device having two components: a needle advancing apparatus slidable longitudinally along a catheter to advance needles into a tissue membrane, such as a blood vessel wall, around an opening in the membrane; and, a suture retrieval assembly insertable through the catheter beyond a distal side of the tissue membrane. The needle advancing apparatus advances suture through the tissue wall. The suture retrieval assembly grabs the suture on the distal side of the tissue membrane for extraction thereof through the opening in the tissue membrane. A method for suturing a membrane beneath the patient's skin is also disclosed.

Such mechanical approaches tend to require precise positioning within the tissue tract, typically provide point (instead of a continuum of tissue purchase) support, and lead to permanent foreign-body implants that interfere with subsequent catheterization at the same vascular site. Additionally, a purely mechanical support of the wound could lead to implanting substantially non-absorbable foreign material that provides only point-support to the wound lips. In addition, purely mechanical closures still can leave behind open micro-spaces, or small gaps, between the sutures that are not entirely closed.

Various biological approaches to vascular closure are described in U.S. Pat. No. 5,108,421 to Fowler. In the '421 patent, a device and method of closing an incision or puncture in a patient is disclosed. The method includes inserting a vessel plug into the incision or puncture until the distal end of the vessel plug is adjacent to the outer lumen of the blood vessel. The vessel plug is positioned so that it does not obstruct the flow of fluid through the blood vessel or target organ. The precise positioning of the vessel plug in the incision or puncture is accomplished through the use of a balloon catheter or a cylindrical insertion assembly having a proximal plunger member associated therewith.

U.S. Pat. No. 5,021,059 to Kensey et al. describes another biological closure approach. The '059 patent, describes deploying a collagen plug to seal the closure. In order to block the collagen from entering the vessel, a footplate is installed on the interior of the blood vessel. The footplate is held in place with a suture. The approaches described in the '059 patent, however, do not physically approximate the lips of the arteriotomy. The '059 patent describes a system which relies on only the collagen to close the wound. Moreover, the '059 patent requires a guide wire for placement and creates a hole through the sealant during the process.

The tissue tract typically can have jagged or rough walls due to, for instance, a tract that was bluntly dissected, a tract having lots of flaps, or a tract just naturally having an uneven structure. When a closure device, catheter, or other similar device is inserted into such a tissue tract it needs help tracking the device, e.g., advancing the device, through the tissue tract without it getting stuck on the uneven wall surfaces of the tissue tract due to corners or sharp edges of the device. In order to help track the device a guide is used to direct the device through the tract, such as a guidewire, suture, etc.

In all of the above approaches a guidewire or some kind of tracking device is needed to place the closure device in position near the arteriotomy and to avoid getting caught on the uneven walls of the tissue tract when advancing the closure device. Where a biological closure is used, the plug or sealant is typically coupled to a wire for placement adjacent to the arteriotomy such that a central hole through the sealant is provided to receive the wire therethrough.

### SUMMARY

A vascular closure device that includes both a mechanical component and a biological component for sealing an arteriotomy, and method for using such a device, is provided herein. In one aspect, the vascular closure device can be used to close large bore arteriotomies. The mechanical component can first reduce the size of the arteriotomy or close the arteriotomy such that only at least one micro-space or gap remains. A micro-space can be a peripheral extension of the arteriotomy that has a fissure-type of configuration and would be smaller than the arteriotomy, and typically having a fissure appearance or configuration. In one aspect, the mechanical component can be sutures or clips. Next, after mechanical closure, the biological component can cover and fill any spaces, cracks, and/or micro-spaces remaining in the arteriotomy after the arteriotomy has been reduced in size or essentially closed by the mechanical component. Thus, the biological component provides adjunctive closure, e.g., it adds to the closure already provided by the mechanical component.

In another embodiment, a vascular closure device is provided that includes only a biological component for sealing an arteriotomy, which can be deployed over an unsealed arteriotomy, e.g., an arteriotomy that has not been exposed to any type of closure mechanism mechanical or biological, or deployed over a reduced or closed arteriotomy that was closed with a prior biological component. In the latter case, a first biological component would be deployed to seal or reduce the arteriotomy. Thereafter, a second biological component, containing the same or a different component, can be deployed on top of or adjacent to the first to cover and fill any spaces, cracks and/or micro-spaces remaining in the arteriotomy after the first biological component is applied. In yet other embodiments, only a single deployment of a first biological component may be necessary, without need for any other additional closure mechanism.

In one embodiment, a smooth rounded plug or sealant, i.e., the biological component, can be advanced into the tissue tract, after the arteriotomy has been reduced in size or essentially closed by the mechanical component, and the sealant can be placed adjacent the mechanically closed arteriotomy. The sealant can be advanced into position without a guidewire such that the sealant is not coupled to a guide wire and therefore can avoid having a central hole therethrough. In situ, the sealant can transform from a solid structure to a flowable or gel state to fill and cover any cracks, spaces and micro-spaces that may arise from the mechanically closed arteriotomy. The sealant, upon deployment into the tissue tract can reconfigure to the shape of the puncture. In one aspect, the sealant can be a polyethylene glycol (PEG) sealant which cross-links in situ. The sealant can cover and fill micropores arising from the mechanically closed lips of the arteriotomy. The sealant can be cast or molded in situ to match the puncture, fissures, and micropores created between the suture strands in the closed arteriotomy. In another embodiment, the sealant can be spherical in shape which can aid in overcoming issues with positioning accuracy due to the spherical shape fitting into place adjacent the arteriotomy better than a non-spherical sealant shape.

In another embodiment, a firm, plug-shaped sealant can be advanced into the tissue tract to cover a mechanically closed arteriotomy in a blood vessel by positioning the sealant immediately adjacent the closed arteriotomy. The step of advancing the sealant can be performed without a guidewire or suture. The sealant can be released into the tissue tract in close proximity of the closed arteriotomy. Upon contact with physiological fluid present in the tissue tract, the sealant can flow onto and between the mechanically closed arteriotomy and fill at least one micro space created between suture strands in the mechanically closed arteriotomy.

In still another embodiment, the sealant can be held within a lumen of an elongate tubular member, and the step of advancing can be performed by advancing the elongate tubular member into the tract without the guidance of a guidewire.

In yet another embodiment, the step of releasing the sealant can be performed by ejecting the sealant from the distal end of the elongate tubular member. In another embodiment, the sealant can be ejected by relative movement between the sealant and the elongate tubular member.

In another aspect, the sealant can be held fixed with a tamper member slidably disposed within the lumen, and the elongate tubular member can be retracted proximally. The elongate tubular member can comprise a distal end having a closed bias, such that the distal end is biased closed with at least one holding member.

The at least one holding member can comprise a plurality of holding members that collectively form a smooth atraumatic surface protruding from the distal end of the elongate tubular member. In one embodiment, the holding members can have a leaf shape, and in another embodiment, the leaf shape can be triangular.

In still another aspect, the method can further comprise lubricating the tissue tract leading to the arteriotomy. In yet another aspect, a closure apparatus can comprise an elongate tubular member with a lubricious coating having a proximal end, a distal end, and an atraumatic distal tip. The lubricious coating can act to ease insertion of the applicator through the tract and/or scarred tissue. The atraumatic distal tip can be formed at least in part by a plurality of leaves, where each leaf can be biased in an inward direction such that the plurality of leaves collectively form the atraumatic distal tip when the atraumatic distal tip is closed.

A plug can be disposed within the tubular member. An advancer member can be provided for ejecting the plug from the elongate tubular member and through the plurality of leaves. In some embodiments, each leaf may have a triangular shape. The sealant plug may be a solid mass and lumen-less, and/or the sealant plug may be spherical. The sealant may also be designed to be visible under standard catheter lab imaging techniques, such as fluoroscopy and ultrasound.

In still other embodiments, the distal tip of the tubular member can have a smooth, curved shape, a radius, and a height. The radius may be equal to, less than or greater than the height.

The closure device disclosed herein can beneficially provide the primary or adjunctive closure to an arteriotomy without the need for advancing the device over a guidewire or other tracking device. The closure device can be provided with an atraumatic tip that, due to its rounded shape, can advance through an uneven tissue tract without getting stuck or hung up on the walls of the tissue tract. In addition, due to the lack of a guidewire or other tracking device, the sealant does not need to be coupled to a wire or suture. Thus, the sealant can avoid having a central hole placed therethrough providing for integrity of the sealant. The sealant itself can be provided in a rounded or spherical shape which is beneficial for sealant placement. The rounded shape can allow the sealant to migrate toward the arteriotomy due to its shape and the characteristic of the sealant. Where the arteriotomy is first closed with a mechanical means, such as a suture, the subsequently placed sealant or adjunctive closure can utilize the mechanically closed or sutured arteriotomy as a locating means to help guide the sealant into place. The sealant, through amorphic expansion once exposed to the physiological fluids will expand and find the arteriotomy and its mechanically closed portion.

Moreover, the closure device described herein is further beneficial because it can be used and inserted into the tissue tract after all of the procedural instruments and guides have been withdrawn. Since the device described herein does not require a guidewire or other tracking device, once the instruments have been removed, they do not have to be reinserted in order to position the closure device described herein.

### BRIEF DESCRIPTION OF THE DRAWINGS

FIG. 1 illustrates a cross-sectional view of a blood vessel, mechanically closed arteriotomy, and tissue tract leading to the arteriotomy;

FIGS. 2-5 illustrate deployment of a sealant through an elongate applicator into the tissue tract of FIG. 1;

FIG. 6 illustrates a cross-sectional view of the closed arteriotomy after deployment of the sealant, and while the sealant is still present;

FIG. 7 illustrates a cross-sectional view of the closed arteriotomy after the sealant has been absorbed;

FIG. 8A illustrates a perspective view of a distal section of the applicator shown in FIGS. 2-5;

FIG. 8b illustrates a side view of the distal section of the applicator of FIG. 8A;

FIG. 9A illustrates a perspective view of a second embodiment of the distal section of an applicator;

FIG. 9b illustrates a side view of the distal section of FIG. 9A;

FIG. 10A illustrates a perspective view of a third embodiment of the distal section of an applicator;

FIG. 10b illustrates a side view of the distal section of FIG. 10A; and

FIGS. 11A-E illustrate side views of fourth through eighth embodiments, respectively, of the distal sections of applicators.

### DETAILED DESCRIPTION

A closure device and method of use are provided for sealing an arteriotomy without the need of a guidewire or other tracking device to track the closure device through the tissue tract and for placement adjacent the arteriotomy. The device can be provided with an atraumatic tip for easier advancement through a tissue tract and with a rounded or spherical sealant for easier placement and deployment of the sealant adjacent the arteriotomy, while maintaining the sealant integrity by avoiding a central hole for a guidewire or suture. This device for sealing an arteriotomy can be used to seal both small and large bore punctures through tissue.

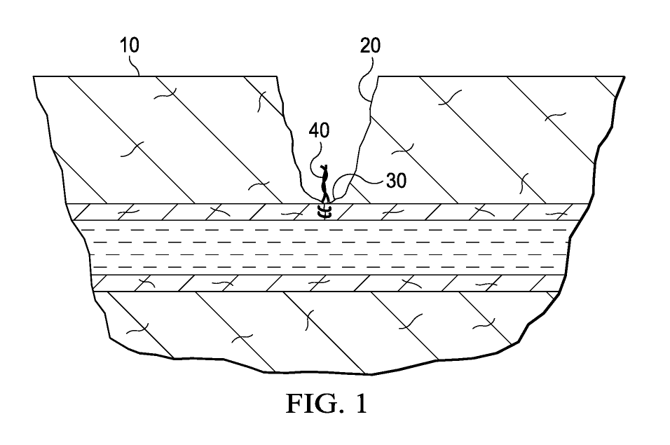

Turning to the drawings, FIG. 1 shows an exemplary embodiment of a tissue tract 20 leading from the exterior surface of the tissue 10 (e.g., a patient's skin) to a closed arteriotomy 30. In this embodiment, the arteriotomy is shown mechanically closed by a suture 40, however, any other type of mechanical closure mechanism can be employed that is appropriate. Suturing devices or means for placing sutures are known in the art, for example, as described in the '078 patent, mentioned herein. However, any other appropriate techniques for mechanically closing the wound lips of an arteriotomy may be used.

Examples of other techniques or devices for mechanically closing the wound lips of an arteriotomy, may include, but are not limited to, clips, sutures, plugs, tacks, sealants, glue, and other devices and implants for closing the arteriotomy or wound lips. Mechanical supports or links can include but are not limited to: sutures (reference numeral 40 of FIG. 1), clips, staples, hooks, scaffolds, disks, balloons, and any other shapes designed to provide temporary or permanent wound support. Additionally, the mechanical component or techniques for approximating the wound lips can further include a temporary implant (PGLA, PLLA, hydrogel, etc) or permanent implant (nitinol, stainless steel, platinum, titanium, etc), and/or techniques for securing a mechanical component (suture, clip). Still further examples of mechanical closure devices and techniques are shown and described in co-pending U.S. patent application Ser. No. 14/179,767, filed Feb. 13, 2014, the disclosure of which is incorporated by reference herein in its entirety.

Thus, the closing of the wound is not intended to be limited by the type of mechanical closure. Moreover, while the term “closed arteriotomy” is used to describe the state of the arteriotomy after mechanical closure, it is to be understood that the arteriotomy may not be completely closed at this point. The arteriotomy is closed such that it is at least reduced in size but may still contain spaces, gaps or micro-spaces where a complete or entire closure has not yet been facilitated. Alternatively, the closed arteriotomy may also mean it is completely closed and that the subsequent biological component is provided to ensure that the completely closed arteriotomy remains that way or as a secondary assurance of that closure.

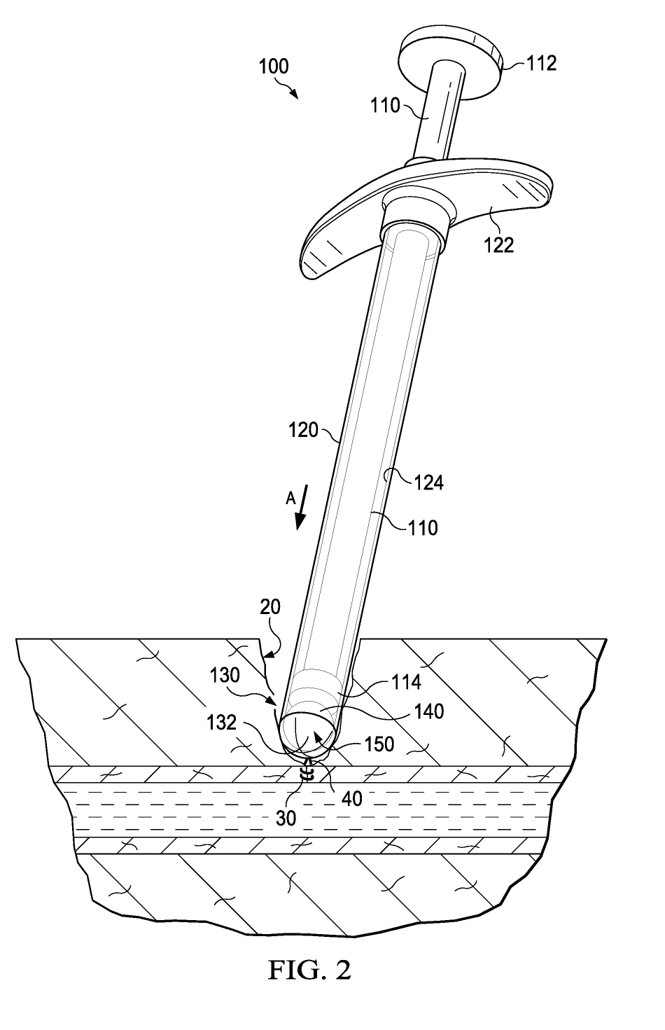

After the arteriotomy of FIG. 1 has been at least reduced in size by the mechanical closure, the biological component can be introduced. In one aspect, the biological component can be introduced into the tissue tract and positioned adjacent the reduced or closed arteriotomy by use of a tool or other device that advances the biological component into place, such as an applicator, as shown in FIG. 2. An applicator 100 can be inserted or advanced into the puncture tract 20 by the user in the direction of arrow A, shown in FIG. 2. The Applicator 100 can be advanced through the tissue tract 20 until a distal end 130 is adjacent the mechanically closed arteriotomy 30. The biological component, such as a biological sealant 140, can be disposed within the applicator 100 at the distal end 130 of the applicator 100 and available for ejection. The applicator 100 does not include or utilize a guidewire to direct or guide the advancement of the applicator 100. Instead of utilizing a guidewire, the applicator can be provided with one or more features that provide for ease of insertion. For example, a lubricious coating can be provided on the outer surface of the applicator shaft 120 for ease of insertion through the tissue tract. In another example, the distal end 130 of the applicator can have a rounded or somewhat rounded tip 150 that can work to advance the applicator without the need for a guidewire. Avoiding the use of a guidewire also allows for the sealant to not be coupled to the guidewire and, as a result, avoids having a hole or central passage through the sealant.

The applicator 100 can have an elongate shaft 120 and a rounded atraumatic tip 150. The tip 150 can have a semi-spherical curvature or other shape appropriate for advancement into and through a tissue tract. The distal tip 150 shown in FIG. 2 can terminate at an atraumatic point, and can radially extend proximally until it smoothly mates with the shaft 120. The atraumatic shape prevents the distal tip 150 of the catheter from engaging or unintentionally catching on the walls of the puncture tract 20. As described further herein the distal tip 150 may have other shapes and is not intended to be limited except as recited in the appended claims. The elongate shaft 120 can further comprise an inner lumen 124 which receives an advancer member or inner tamping member 110 therein. The distal end of the inner member 110 rests adjacent to the sealant 140. The distal end of the inner support 110 can further comprise a contacting-end 114 that can contact the sealant 140, and the contacting-end 114 can be shaped like a disk in one embodiment as shown in FIG. 2, or any other appropriate shape. The inner member 110 can extend through the inner lumen 124 of the shaft 120 and can extend beyond the proximal end of the shaft 120, terminating in a plunger handle 112.

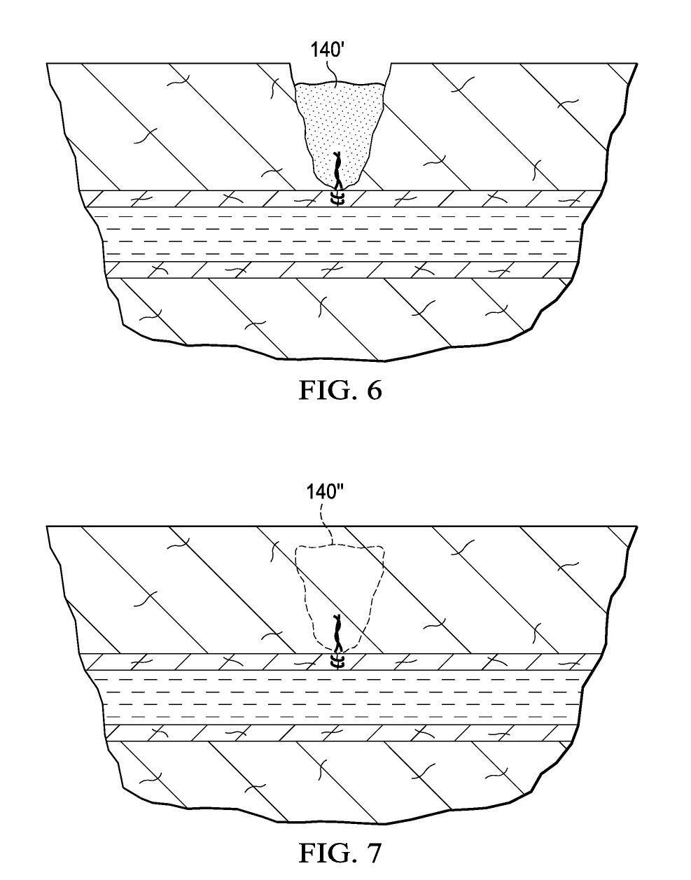

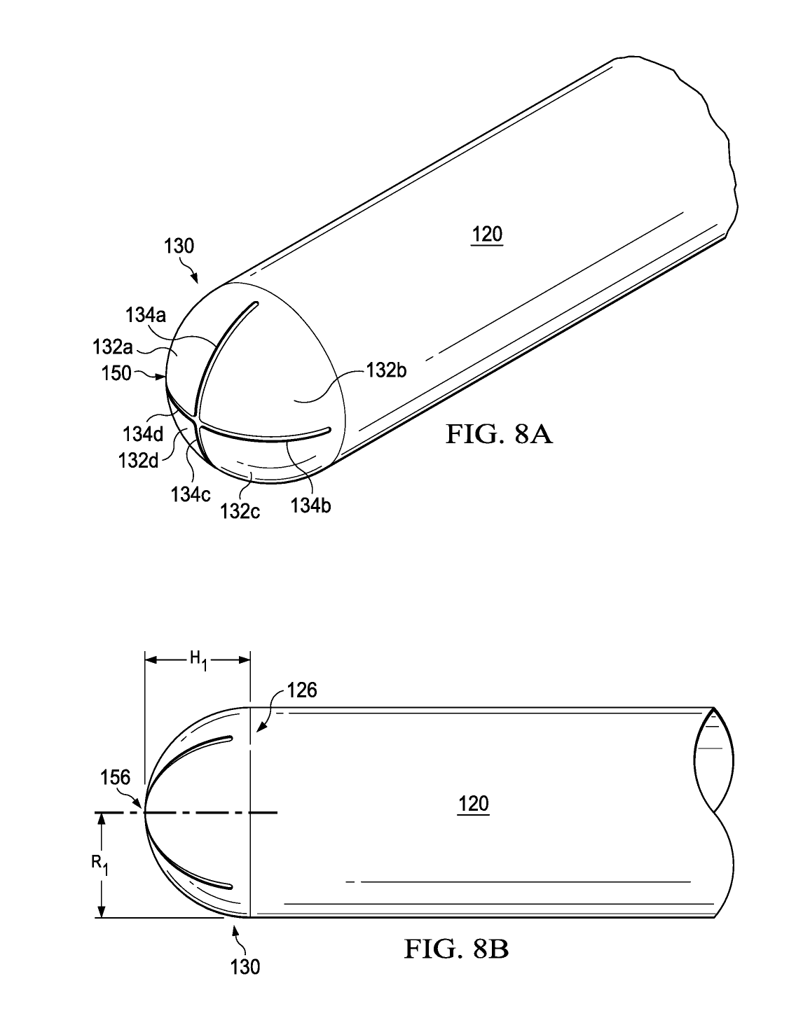

At the distal end 130 of the applicator 100, the tip 150 can be initially provided in a closed position, as shown in FIG. 2. The tip 150 can comprise several panels or leaves 132 that are initially in a closed state enclosing the sealant 140 therein. In turning to FIGS. 8A-8B, a tip 150 is shown with at least four panel members or leaves 132a-d in the closed state. In order to deploy the sealant, e.g., to release it from the internal lumen of the applicator shaft 120, the leaves 132 are shifted from the closed position to an open position, which will be discussed in further detail below. The panels or leaves 132 of the distal tip 150 can comprise more or less panels than the four illustrated in the figures. For example, the distal tip 150 can comprise at least two leaves, at least three leaves, or more than four leaves, depending on the design that is most appropriate for the application.

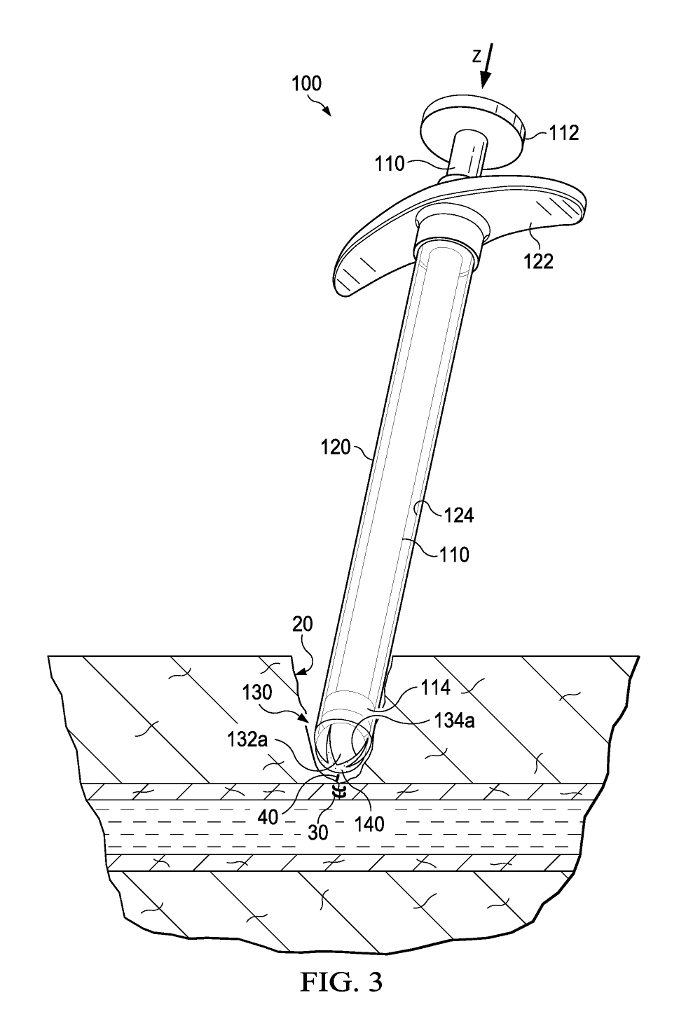

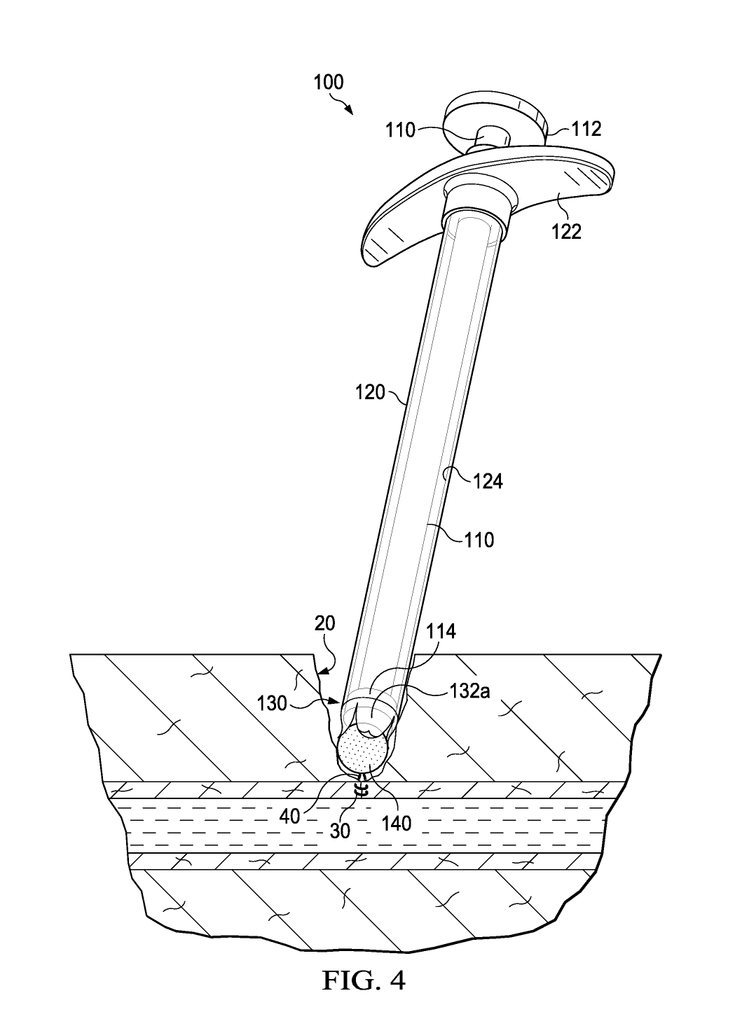

Once the applicator 100 is in position adjacent the closed arteriotomy, the sealant 140 can then be deployed. In one embodiment, a plunger handle 112 or other similar device can be used to shift the inner member 110 in a distal direction, e.g., in a downward direction as shown by arrow Z, and at the same time the applicator shaft 120 and inner tamping member 110 can be moved relative to one another, e.g., such that the inner tamping member 110 moves distally while the shaft 120 moves proximally. As the inner tamping member 110 is being shifted in a distal direction, the contacting-end 114 can further be pressed into contact with the sealant 140, urging the sealant 140 in a distal direction and into contact with the rearward surface of the leaves 132, as shown FIGS. 3-4. In particular, with reference to FIG. 3, the leaves 132a-d can be deflected from their biased closed position as the sealant 140 is pushed against the rearward surface of the leaves 132a-d. As the leaves 132a-d are further shifted from the closed position into an open position (and the tip 150 is deflected to an open position), the sealant 140 can be released and/or ejected from the inner lumen 124 of the elongate shaft 120 and into the tissue tract 20, as can be seen in FIG. 4. The outer shaft 120 can be further retracted while the sealant 140 can be held in place with the advancer member 110. The leaves 132a-d can be substantially deflected or biased outwardly at this point to provide an opening for the sealant 140 to pass through. Consequently, the sealant 140 can be ejected adjacent the closed arteriotomy and can begin reacting with the physiological materials of the tract 20. As the sealant 140 begins to react with the physiological materials of the tract 20 it can also begin to surround or incorporate portions of the sutures 40 or other mechanical closure device.

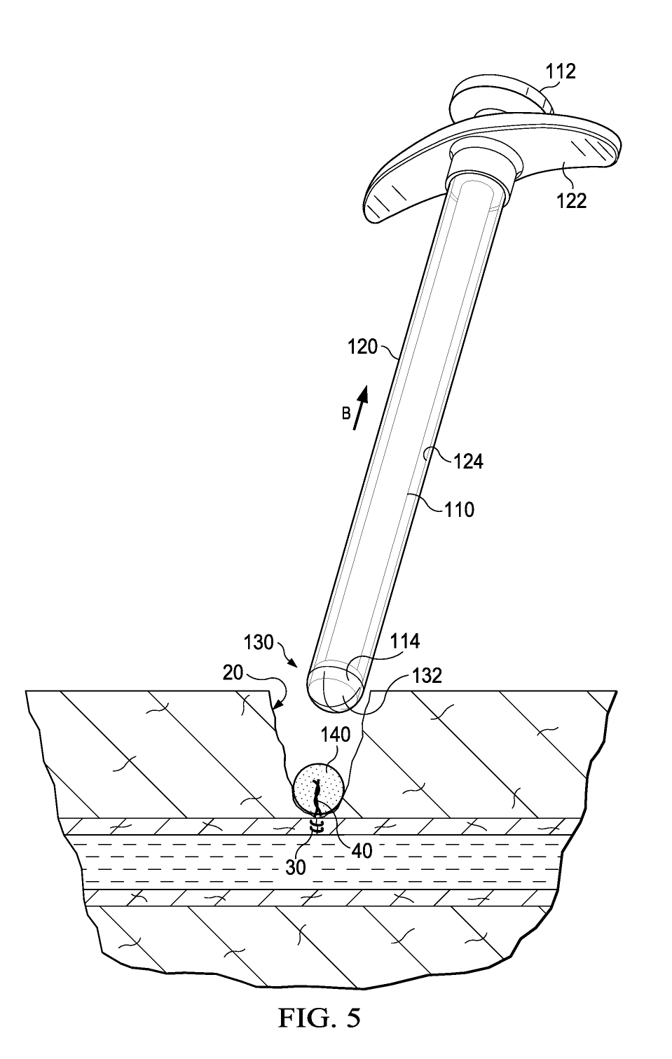

Once the sealant 140 is fully deployed, the applicator device 100 can be withdrawn from the tissue tract 20 in the direction of arrow B, as shown in FIG. 5. As shown in FIG. 5, after the sealant has been fully deployed, the leaves 132a-d can be returned to the closed position. The contacting-end 114 of the advancer member 110 typically does not extend beyond the transition point where the distal end 126 of the shaft 120 meets the tip 150, even when the plunger handle 112 has been fully depressed (as in FIG. 5), such that once the sealant 140 is ejected from the inner lumen 124, there is no other device or object pressing against the rearward surface of the leaves 132 to bias the leaves 132 in the open position. Thus, the leaves 132 are no longer shifted outward by the force of the sealant being pressed in the distal direction and the leaves 132 are shifted back again into their resting position, i.e., the closed position.

After the sealant 140 has been deployed, it can continue to react with the physiological fluid in the tissue tract and adjacent the closed arteriotomy, as shown in FIG. 6. Preferably, plug 140 can absorb or otherwise react with the environment to change shape and properties which better serve to close the arteriotomy. The sealant can transform from a first relatively firm state 140 to a second more gelatinous state 140′. The reacted sealant 140′ can combine with wound coverage of the mechanical closure to reduce or fully close the reduced or approximated arteriotomy.

After a given period of time, the sealant 140′ is absorbed into the body, as indicated in FIG. 7. The mechanically approximated wound lips 30 are shown after the sealant has been absorbed. Tissue volume 140″ is indicated where the sealant has been absorbed. Consequently, the tissue tract or puncture is closed, preventing blood leak and oozing.

The sealant is preferably made of a biological component or material that is absorbed when it is placed in the puncture tract. Examples of sealant materials may include polyethylene glycol (PEG), collagens, and hydrogels already cross-linked or that cross-link in situ. In certain embodiments, the biological component can be solid or injectable, and it can comprise a plurality of different biological forms. In other embodiments, the biological component can be a smart moiety that selectively binds to tissue or molecular domains in proximity of the wound; a moiety that infiltrates the wound and wound surrounding tissue such as to seal the wound; a composition of infiltrating and selectively binding moieties; and/or incorporate other materials/components that provide enhancement to the biological moiety such as reinforcement, visibility, expansion, etc.

Optionally, the sealant 140 may be further tamped by advancing support member 110. This optional step serves to urge the sealant 140 against the outside surface of the arteriotomy lips, causing the gelatinous sealant to further fill micro-spaces or gaps left unclosed by the mechanical bond, and generally fill a section of the tissue tract 20.

In still other embodiments, the biological components can be polymers, proteins, other molecules, or a conjugation/combination of types of these components, individually or collectively, designed to provide continuous wound coverage. Non-limiting examples of biological sealants can include biodegradable gels such as PEG, and collagens. The sealant may have a first plug shape that is relatively solid-like, and transform to a flowable expandable gel, for example.

Examples of materials for the catheter components include medical grade plastics such as polyether ether ketone (PEEK), nylons, polyether block amide (PEBA), and other similar compounds. Such materials may be injection molded, extruded, or otherwise machined and built to shape.

In the illustrated embodiments, each leaf is shown shaped as a triangular leaf and, collectively, the plurality of leaves can mate at a distal point, by contacting each other at their distal ends, forming a smooth enclosure. However, the leaf members may have other shapes. Moreover, the leaves may overlap with each other or mate at an edge without overlap or any other appropriate orientation that allows for the tip 150 to maintain a closed position. Additionally, the leaf shapes can have another shape besides triangular; they can be rectangular, square, etc., or any shape that is appropriate to form the tip 150.

In the embodiment shown in FIG. 8A, the distal section 130 of an applicator 100 is shown having a smooth, semi-spherical shape. There can be four panels or leaves 132a, 132b, 132c, 132d defined by slits 134a, 134b, 134c. 134d. For example, slit 134a can be positioned between panels 132a and 132b, slit 134b can be positioned between panel 132b and 132c, and so on, such that at least one edge of each panel abuts the slit. As discussed herein, the leaves 132a-d can be biased closed to hold the sealant (not shown) within the lumen 124 of the shaft 120. Upon deployment of the sealant by pushing down on the plunger handle 112, the advancer member 110 is advanced in a distal direction and may then urge the sealant in the distal direction, displacing the biased leaves 132a-d outwardly, and thus ejecting the sealant.

The curvature of the tip 150 can further be defined by the ratio R₁:H₁, whereby H₁ is the height of the tip defined by the portion of the tip 150 from the distal end 156 up to the transition point where the tip 150 meets the distal end 126 of the shaft 120, and R₁ is the radius of the catheter shaft, as shown in FIG. 8B. The size/dimension can be determined by the size of the femoral artery (e.g., determined by the use). The R:H ratio can define varying tip shapes: for example, an R:H ratio equaling 1 produces a perfect hemisphere tip, while R:H<1 can represent an elongated/more oblong/pointed tip shape, or R:H>1 can represent a squat/blunted/flattened tip shape. A ratio of R:H can range from about 0.30 to about2.50. In the embodiment shown in FIG. 8B, H₁ can be about equal to R₁. In other embodiments, R₁ can range from about 1 mm to about 4 mm, and H₁ can have corresponding values such that the ratio R₁:H₁ can be chosen within the range from 0.30 to about 2.50.

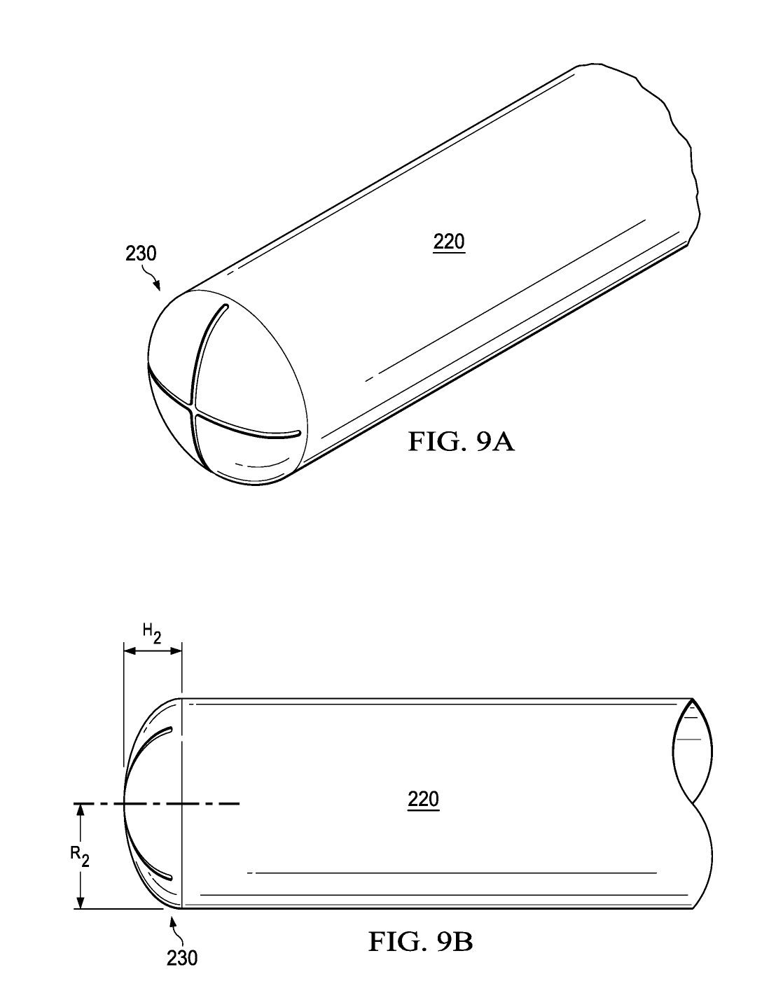

In FIGS. 9A-9B, an alternate embodiment is provided of the tip of the applicator. The applicator shaft 220 shown in FIGS. 9A-9B is similar to that shown in FIGS. 8A-8B except that the distal section 230 has a tip that has a more squat, blunted shape. A ratio of R₂:H₂ can range from 1.0 to 2.50. In particular, R₂ can have a range from about 1 mm to about 4 mm, and H₂ can have corresponding values such that the desired ratio is achieved. In another aspect, H₂ can have a smaller value than R₂ such that the tip provides the shape as shown in FIG. 9B.

Without being bound by theory, the flattened/squatter tip shape shown in FIGS. 9A-9B can provide a more flush contact with the mechanically closed lips of the arteriotomy. In addition, a squatter profile may have less risk of the tip and/or sealant protruding into the blood vessel.

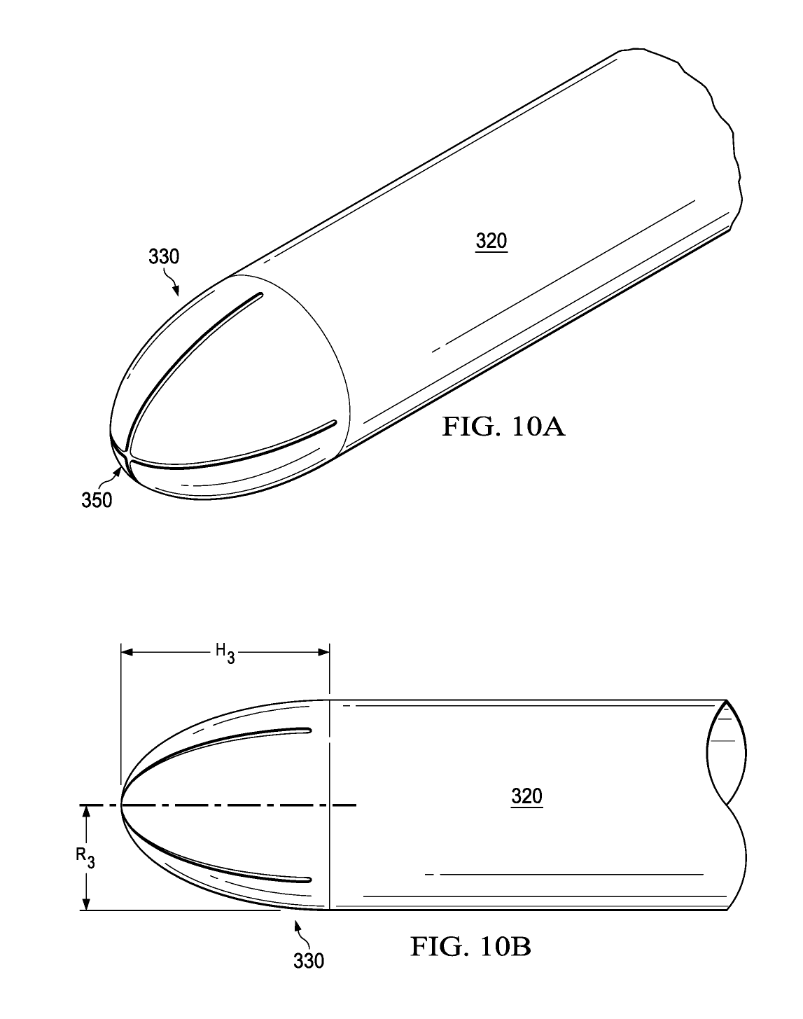

In still another alternate embodiment, a distal section 330 at the distal end of the shaft 320 is shown with a more elongated tip 350, as shown in FIGS. 10A-10B. The applicator 320 is similar to that shown in FIGS. 8A-8B except that the tip at the distal section 330 has a longer, more oblong and pointed shape. In particular, R₃ can range from about 1 mm to about 4 mm and H₃ can have corresponding values such that a ratio of R₃:H₃ can range from 0.30 to 0.95. Without being bound to theory, the more pointed curvature of the tip as shown in FIGS. 10A-10B can have the benefit of facilitating navigating through a puncture tract leading to an arteriotomy. There can be less of a tendency to catch or frictionally interact with the walls of the puncture than the more square counterpart.

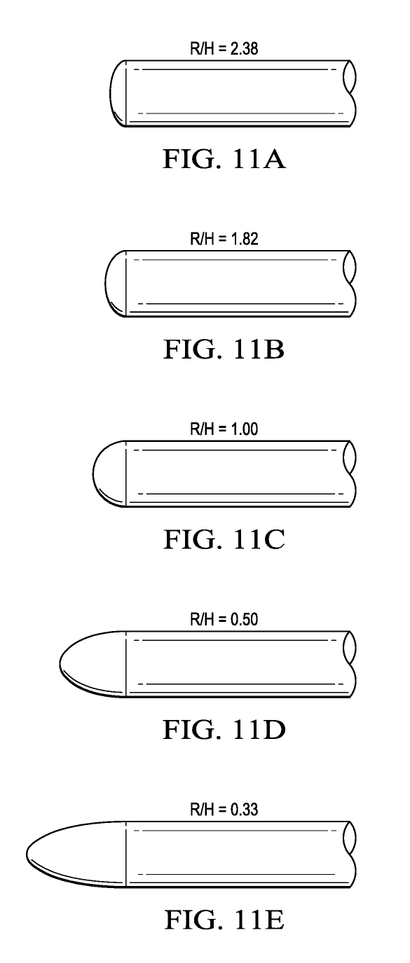

Still other alternate embodiments of the distal tip shape of the shaft can be shown in FIGS. 11A-E. Alternate tip shapes are shown having varying ratios. For example, a ratio of R:H having a value of 2.38 has a very squat, short tip end, as shown in FIG. 11A. On the other hand, a ratio of R:H of 0.33 can result in an elongated, pointy tip as shown in FIG. 11E. Other shapes of the tip for other R:H values are illustrated in the remaining FIGS. 11B-11D.

In one aspect, the shape of the sealant used in combination with the applicator 100 can be a spherical-shaped sealant. A spherical sealant can overcome the positioning accuracy issues when a sphere is deployed in the tissue tract. Due to its rounded shape, the sealant can migrate toward the arteriotomy easier than a sealant that is less round and may have edges or corners that can get stuck on the tissue tract walls. In addition, the sealant can have any other shape that is appropriate for deploying the sealant through the tip 150 and for positioning the sealant in the tissue tract adjacent the arteriotomy. Moreover, the sealant in these embodiments does not contain a central hole or other passage therethrough due to avoiding a central guidewire that would need to pass through the sealant. Thus, the sealant can avoid a having a portion removed.

Embodiments include any one or combination of the devices, methods, kit of apparatuses, systems, and implants as described herein. Moreover, while a method of providing an adjunctive biological sealant to a closed arteriotomy having sutures was described above, a similar process can be utilized where the arteriotomy was previously closed with another mechanical means or instead by a biological means, such that it would result in a biological closure on top of a biological closure. Alternatively, the above method can be provided on an arteriotomy that has not been closed at all yet, such that the sealant as disclosed herein would be provided as the first or primary closure means.

Other modifications and variations can be made to the disclosed embodiments without departing from the subject disclosure. Although FIGS. 1-11 illustrate percutaneous access to a blood vessel, the methods and devices are not intended to be so limited except as where specifically recited in the claims. The methods and devices may be utilized in procedures other than percutaneous procedures such as, for example, direct or open surgeries.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the range. Also, it is contemplated that any optional feature of the variations described may be set forth and claimed independently, or in combination with any one or more of the features described herein.

It is to be understood that the method and apparatus disclosed herein is not limited to particular variations set forth herein as various changes or modifications may be made to the method and apparatus described herein and equivalents may be substituted without departing from the spirit and scope disclosure. As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. In addition, many modifications may be made to adapt a particular situation, material, composition of matter, process, process act(s) or step(s) to the objective(s), spirit or scope of the method and apparatus described herein. All such modifications are intended to be within the scope of the claims made herein.

What is claimed is:

1. A method for adjunctively sealing a puncture containing a mechanical closure, the puncture extending through tissue to a body lumen, said method comprising:
advancing a plug-shaped sealant into a tissue tract until the sealant is positioned in close proximity to the puncture having the mechanical closure, wherein the step of advancing the sealant is performed without a guidewire or suture; and
releasing the sealant into the tissue tract in close proximity of the puncture, wherein upon contact with physiological fluid present in the tissue tract, the sealant infiltrates and expands onto and around the puncture having the mechanical closure.

2. The method of claim 1, wherein the step of advancing the sealant further comprises advancing an elongate tubular member comprising a lumen, where the elongate tubular member is guideless, and the sealant is disposed within the lumen.

3. The method of claim 2, wherein the elongate tubular member comprises a distal end having a rounded shape, where the distal end of the elongate tubular member is biased closed with at least one panel.

4. The method of claim 3, wherein the step of releasing the sealant further comprises ejecting the sealant from the distal end of the elongate tubular member.

5. The method of claim 3, wherein the at least one panel comprises a plurality of panels that collectively form a smooth atraumatic tip protruding from the distal end of the elongate tubular member.

6. The method of claim 5, wherein the atraumatic tip comprises a radius and a height.

7. The method of claim 6, wherein the radius is greater than the height.

8. The method of claim 2, further comprising advancing a tamping member slidably disposed within the lumen, while the elongate tubular member is retracted proximally.

9. The method of claim 8, wherein the step of releasing the sealant comprises ejecting the sealant from the distal end of the elongate tubular member by advancing the tamping member and contacting the sealant with the tamping member.

10. The method of claim 1, further comprising lubricating the tissue tract leading to the puncture.

11. The method of claim 1, wherein the sealant is delivered as a spherical-shaped sealant.

12. A vascular closure device for sealing a puncture through tissue comprising:
an elongate tubular member having a proximal end, a distal end, and a rounded distal tip, said rounded distal tip formed at least in part by at least one panel member, the at least one panel member being biased in an inward direction such that the at least one panel member forms part of the rounded distal tip when the rounded distal tip is in a closed position, wherein the elongate tubular member is guideless;
a plug-shaped sealant disposed within the elongate tubular member; and
an advancer member for ejecting the sealant from the elongate tubular member and through the at least one panel member, and
wherein the at least one panel member, when in the closed position, provides a force opposing advancement of the sealant from the elongate tubular member.

13. The device of claim 12, wherein the sealant is spherical.

14. The device of claim 12, wherein the sealant is a solid mass and lumen-less.

15. The device of claim 12, wherein the rounded distal tip of the elongate tubular member has a curved shape, a radius, and a height.

16. The device of claim 15, wherein the radius is greater than the height.

17. The device of claim 15, wherein the rounded distal tip of the elongate tubular member has a ratio of radius to height, R:H, ranging from about 0.33 to about 2.38.

18. The device of claim 12, further comprising a handle, and wherein the handle is disposed at the proximal end of the elongate tubular member.

19. The device of claim 12, wherein the at least one panel member comprises a plurality of panel members, each panel member being biased in an inward direction such that the plurality of panel members collectively form the rounded distal tip when the rounded distal tip is in the closed position.

20. The device of claim 19, wherein each panel member has a triangular shape.
